(19) United States
(12) Patent Application Publication
(10) Pub. No.: US 2026/0000529 A1
(43) Pub. Date: Jan. 1, 2026

(54) ORTHOSIS DEVICES AND METHODS RELATING TO THE SAME

(71) Applicant: Ralph C. Bertonaschi, Olmsted Township, OH (US)

(72) Inventor: Ralph C. Bertonaschi, Olmsted Township, OH (US)

(21) Appl. No.: 18/760,481

(22) Filed: Jul. 1, 2024

(51) Int. Cl. A61F 5/058

(52) U.S. Cl. CPC A61F 5/05875

(57) ABSTRACT

Orthosis devices and methods for stabilizing a limb of an individual comprising a first member having a first inner surface that is configured to receive a first portion of a limb; a second member having a second inner surface that is configured to receive a second portion of the limb; and a coupler arrangement that couples the first member to the second member such that the orthosis device is transitionable between an open position and a closed position.

### TECHNICAL FIELD

This invention pertains to a splint and more specifically to an orthosis device with a rigid outer shell and an inner impression material. The orthosis device is designed to be positioned partially or fully about a limb, toe, or finger in order to treat and heal injuries and associated discomfort, particularly to fingers and toes, and more particularly to an orthosis device that is designed to be positioned partially or fully about a limb, toe, or finger in order to treat and heal injuries and associated discomfort, particularly to fingers and toes, and is also configured to form and set circumferentially about such limb, toe, or finger in order to treat and heal injuries and associated discomfort, particularly to fingers and toes.

### BACKGROUND OF DISCLOSURE

Human bones can be broken and require time to heal. When a bone in a limb is broken, it is typically immobilized during the healing process. Such immobilization may be done by placing the limb in a plaster cast. A plaster cast is beneficial because it offers a stable environment to keep bones set for ideal healing. A plaster cast is limited though because, in the event of misalignment, the plaster cast must be completely removed and redone, which is a time intensive process. Also, it would be beneficial to be able to remove the cast during the healing process in the event that other medical services need to be performed. What is needed is a cast that provides stability but is easily removable and can be reapplied at will.

In addition, problems are amplified when the person has a broken finger or toe. Human fingers and toes, also referred to as digits, are involved in most of one's daily purposeful tasks, and injury or disability to any of these appendages can significantly affect one's ability to manipulate objects and perform even the most simple or complex tasks. Fingers are susceptible to common injuries such as fractures, sprains, strains, and dislocations to name a few, and certain ailments, for example, arthritis, tendonitis, and joint instability. Injuries can occur, for example, from digits getting slammed in doors, stepped on, crushed, or hit with great force and may eventually heal but cause future discomfort.

Finger splints, also referred to as finger orthoses, are medical devices designed to immobilize a finger and hold it in position to protect and support said finger following injury or ailment. This apparatus is often used to help heal bone fractures, damaged tendons or ligaments, sprains, or other pathologies while attempting to reduce additional stress to the injury.

Finger splints in the current marketplace come in a variety of sizes, shapes, and designs and are specific to which finger or type of ailment or injury they treat. Some common finger splints include gutter splints that cover the phalanges on the sides and the palm, two-finger splints that require two fingers fashioned together for support, and stack splints which cover the middle and distal phalanxes and the tip of the finger. Some types of splints also begin coverage over the forearm, wrist, and fingers. Factors such as level of inflammation, severity of injury and type, immobilization level, and size and fit are important elements to determine when deciding what type of device may be required. This determination is of severe importance as an improper method of immobilization may prevent an injury from regaining full range of motion or lead to permanent finger deformities.

Oftentimes, finger splints or orthoses are quite small and lack protective support from the possibility of additional injuries during treatment. If the injury is over-exposed and lacking protection, any minor bump, vibrational force, and/or excessive movement could prevent the injury from properly healing, cause additional damage, or beset significant, detrimental effects to the treatment course. In transverse, some orthoses are overly bulky, connecting multiple fingers together at times, and sometimes secured up the forearm, and often interfere with complete functional use of the hand. Such an overly supported device may impede the daily function and responsibilities of an injured person and make simple tasks quite difficult. Common to the marketplace finger splints also lack customizability when considering the varying sizes and shapes of injured fingers.

Accordingly, what is needed is an orthosis device that is customizable in size and designed to distinctively conform to the unique characteristics of an injured human limb, finger, or toe using an impressionable inner material, while providing stabilization and immobilization.

### SUMMARY OF DISCLOSURE

The present disclosure is directed to orthosis devices and methods that are customizable in size and designed to distinctively conform to the unique characteristics of an injured human limb, finger, or toe using an impressionable inner material, while providing stabilization and immobilization.

In one non-limiting aspect of the present disclosure, there is provided an orthosis device having a first member that has a first inner surface that is configured to receive a first portion of a limb; a second member that has a second inner surface that is configured to receive a second portion of the limb; and a coupler arrangement that couples (e.g., pivotally couples, non-pivotally couples, etc.) the first member to the second member such that the orthosis device is transitionable between an open position and a closed position. The coupler arrangement is configured to retain the first and second members together when in the closed position. In the closed position, one or more edges of first and second members are maintained in contact with one another or held in close proximity with each other by the coupler arrangement. In one non-limiting arrangement, the orthosis device encapsulates 80-100% (and all values and ranges therebetween) the outer surface of the limb in the region of the damaged or injured limb.

In another and/or alternative non-limiting aspect of the present disclosure, there is provided an orthosis device further comprising a filler material that is configured to conform to a shape of the limb, wherein the filler material is disposed on the first inner surface of the first member and/or the second inner surface of the second member. In some non-limiting embodiments, the filler material includes, but is not limited to, silicon, urethane, latex rubber, epoxy resin, polyethylene foam, or combinations thereof. In another non-limiting embodiment, the filler material is optionally a settable or curable material wherein the filler material is easily conformable or the shape of a portion or of all of a limb prior to the filler material being fully set or cured, and after the filler material is set or cured, the filler material partially or fully maintains its set or cured shape. In another and/or additional embodiment, the filler material is optionally a compressible material after the filler material is dried, set and/or cured such that when the filler material is at least partially compressed about a limb when the first and second members are in the closed position, the filler material can compress, where needed, to better confirm about the limb.

In another and/or alternative non-limiting aspect of the present disclosure, the first and second members are optionally pivotally connected together by a first coupler that includes a hinge. The first coupler is configured to maintain a portion (e.g., 10-99.99% of the longitudinal length and all values and ranges therebetween) or all of the longitudinal length of an edge or edge region of the first and second members together or in generally constant spacing from one another when the orthosis device in in both the open and closed position. The hinge is configured to extend partially (e.g., 10-99.99% of the longitudinal length and all values and ranges therebetween) or fully along a longitudinal length of the orthosis device. In certain non-limiting embodiments of the present disclosure, the hinge is a living hinge, a piano hinge, a butt hinge, or a leaf hinge. One or more portions of the hinge can be a) integrally formed with one or more components of the orthosis device (e.g., living hinge, etc.), b) molded to one or more components of the orthosis device, and/or c) adhesively connected or otherwise connected to one or more components of the orthosis device. In another and/or alternative non-limiting aspect of the present disclosure, the hinge includes an adhesive that is at least partially deposited on a contact surface of the hinge, wherein the adhesive is a repositionable adhesive, a cohesive adhesive, or combinations thereof. The hinge, when used, can be a permanent hinge or can be a hinge that is configured to be separatable and reconnectable. The hinge, when used, maintains a portion (e.g., 10-99.99% of the longitudinal length and all values and ranges therebetween) or all of the longitudinal length of an edge or edge region of the first and second members together or in generally constant spacing from one another when the orthosis device in in both the open and closed position.

In another and/or alternative non-limiting aspect of the present disclosure, there is provided an orthosis device further comprising a second coupler that includes one or more fasteners that are configured to secure the first member to the second member when the orthosis device is in the closed position, wherein the second coupler is spaced from the first coupler (e.g., positioned opposite the first coupler, etc.). In certain non-limiting embodiments of the present disclosure, the second coupler includes one or more of clasps, snaps, buttons, magnets, hooks, adhesives, hook and loop fastener, mushroom connectors, clamps, zipper, or combinations thereof. The second coupler maintains a portion (e.g., 10-99.99% of the longitudinal length and all values and ranges therebetween) or all of the longitudinal length of an edge or edge region of the first and second members that is at a location that is spaced from the first coupler together or in generally constant spacing from one another when the orthosis device is in the closed position. When the second coupler is disconnected or disengaged, the edge or edge region of the first and second members that the second coupler is located enables such edge or edge region of the first and second members to be moved apart from one another when the orthosis device moves from the closed to the open position.

In another and/or alternative non-limiting aspect of the present disclosure, the first and second members include a first coupler that is absent a pivot connection together. In one non-limiting arrangement, the first and second members includes first and second couplers that form a releasably snap arrangement wherein the first and second members can be releasably snapped together. In another and/or alterative non-limiting arrangement, the first and second couplers on the first and second members form releasable connections. The first and second couplers can be in the form of an adhesive, hook and loop fasteners, mushroom connectors, clamps, zipper, magnets, buttons, hooks, clasps, etc. In another and/or alternative arrangement, the first and second members are releasably connected together by one or more straps, tape, sleeves, covers, etc. that are positionable about a portion or all of the other surface of the first and/or second members. Such one or more straps, tape, sleeves, covers, etc. can be a substitute for the first and/or second couplers, or be used in combination with the first and/or second couplers.

In another and/or alternative non-limiting aspect of the present disclosure, the first and/or second couplers are positioned one and/or near a side edge of the first and/or second members. The first and/or second couplers can be positioned along 5-100% (and all values and ranges therebetween) the longitudinal length of the one or more of the side edges or edge regions of the first and/or second members. In one non-limiting arrangement, the first and/or second couplers cause one or more side edges abutted against one another when the orthosis device is in the closed position.

In another and/or alternative non-limiting aspect of the present disclosure, there is provided an orthosis device optionally further comprising one or more markers formed at predetermined locations on the first member, the second member, or the first and second members, wherein the one or more markers can be configured to guide an individual in removing a portion of the orthosis device. The one or more marker can be colored regions and/or physical markers.

In some non-limiting embodiments of the present disclosure, the first member and the second member are configured to form an outer shell when the orthosis device is in the closed position, wherein the outer shell is substantially cylindrical, elliptical, cuboidal, spherical, or triangular; however, it will be appreciated that other or additional shapes can be used.

In another and/or alternative non-limiting aspect of the present disclosure, there is provided a method for stabilizing a limb of an individual, the method comprising the steps of a) providing an orthosis device that includes a first member that has a first inner surface, a second member that has a second inner surface, and a coupler arrangement that releasably couples the first member to the second member; b) positioning the orthosis device in an open position; c) positioning a portion of the limb on the first inner surface of the first member and/or on the second inner surface of the second member; d) positioning the first and second members with respect to one another to enable the coupler arrangement to releasably connect the first and second members together; and e) releasably connecting the first and second members together by the coupler arrangement to position the orthosis device into a closed position and to cause the first and second members to partially or fully encapsulate the injured position of the limb.

In another and/or alternative non-limiting aspect of the present disclosure, there is provided a method for stabilizing a limb of an individual further comprising the step of, prior to positioning the portion of the limb on the first inner surface of the first member and/or on the second inner surface of the second member, depositing a filler material on the first inner surface of the first member and/or on the second inner surface of the second member, wherein the filler material is configured to conform to a shape of the limb. In certain non-limiting embodiments of the present disclosure, the filler material includes, but is not limited to, silicon, urethane, latex rubber, epoxy resin, polyethylene foam, or combinations thereof.

In another and/or alternative non-limiting aspect of the present disclosure, there is provided a method for stabilizing a limb of an individual further comprising the step of, releasably securing the orthosis device in the closed position by use of the coupler arrangement, wherein the coupler arrangement includes first and second couplers. The first and second couplers can be the same or different. In one non-limiting embodiment, the first coupler is a hinge type connection and the second coupler is one or more of clasps, snaps, buttons, hooks, adhesives, zipper, magnet, hook and loop fastener, mushroom connectors, adhesive, or combinations thereof. In another non-limiting arrangement, the orthosis device optionally includes first coupler in the form of a hinge connection on the first and second members and the second coupler is spaced from the hinge connection (e.g., opposition side of the first and second members from the hinge connection, etc.).

In another and/or alternative non-limiting aspect of the present disclosure, there is provided a method for stabilizing a limb of an individual wherein the coupler arrangement includes a hinge, and wherein the hinge extends partially or fully along a longitudinal length of the orthosis device. In one non-limiting embodiment of the present disclosure, the hinge is a living hinge, a piano hinge, a butt hinge, or a leaf hinge. In another and/or alternative non-limiting aspect of the present disclosure, the hinge includes an adhesive at least partially deposited on a contact surface of the hinge, wherein the adhesive is a repositionable adhesive, a cohesive adhesive, or combinations thereof.

In another and/or alternative non-limiting aspect of the present disclosure, there is provided a method for stabilizing a limb of an individual further comprising the step of, forming one or more markers at predetermined locations on the first member, the second member, or the first and second members to guide an individual in removing a portion of the orthosis device.

In another and/or alternative non-limiting aspect of the present disclosure, there is provided a method for stabilizing a limb of an individual further comprising the step of, subsequent to positioning the orthosis device into the closed position, forming an outer shell that is substantially cylindrical, elliptical, cuboidal, spherical, or triangular.

One non-limiting object of the present disclosure is the provision of an orthosis device that includes a) first member having a first inner surface that is configured to receive a first portion of a limb; (b) a second member having a second inner surface that is configured to receive a second portion of the limb; and (c) a coupler arrangement that couples the first member to the second member such that the orthosis device is transitionable between an open position and a closed position.

Another and/or alternative non-limiting object of the present disclosure is the provision of an orthosis device wherein the coupler arrangement includes a hinge that is use to pivotally connect together the first and second members.

Another and/or alternative non-limiting object of the present disclosure is the provision of an orthosis device that further comprises a filler material that is configured to conform to a shape of the limb, wherein the filler material is disposed on the first inner surface of the first member and/or the second inner surface of the second member.

Another and/or alternative non-limiting object of the present disclosure is the provision of an orthosis device wherein the filler material includes silicon, urethane, latex rubber, epoxy resin, polyethylene foam, or combinations thereof.

Another and/or alternative non-limiting object of the present disclosure is the provision of an orthosis device that wherein the coupler arrangement includes a hinge, and wherein the hinge extends at least partially or fully along a length of the orthosis device.

Another and/or alternative non-limiting object of the present disclosure is the provision of an orthosis device wherein the hinge is a living hinge, a piano hinge, a butt hinge, or a leaf hinge.

Another and/or alternative non-limiting object of the present disclosure is the provision of an orthosis device wherein the hinge includes an adhesive at least partially deposited on a contact surface of the hinge, wherein the adhesive is a repositionable adhesive, a cohesive adhesive, or combinations thereof.

Another and/or alternative non-limiting object of the present disclosure is the provision of an orthosis device further comprising one or more markers formed at predetermined locations on the first member, the second member, or the first and second members, wherein the one or more markers guide an individual in removing a portion of the orthosis device.

Another and/or alternative non-limiting object of the present disclosure is the provision of an orthosis device that further comprising first and second couplers that are configured to secure the first member to the second member when the orthosis device is in the closed position, and wherein the second coupler is positioned opposite the first coupler.

Another and/or alternative non-limiting object of the present disclosure is the provision of an orthosis device wherein the second coupler includes one or more of clasps, snaps, buttons, magnets, zipper, hook and loop fastener, mushroom connector, hooks, adhesives, or combinations thereof.

Another and/or alternative non-limiting object of the present disclosure is the provision of an orthosis device wherein the first member and the second member are configured to form an outer shell when the orthosis device is in the closed position, wherein the outer shell is substantially cylindrical, elliptical, cuboidal, spherical, or triangular.

Another and/or alternative non-limiting object of the present disclosure is the provision of a method for stabilizing a limb of an individual, the method comprising the steps of (a) providing an orthosis device, comprising: (i) a first member having a first inner surface; (ii) a second member having a second inner surface; and (iii) a coupler arrangement that couples the first member to the second member; (b) positioning the orthosis device in an open position; (c) positioning a portion of the limb on the first inner surface of the first member; and (d) by way of the coupler arrangement, positioning the orthosis device into a closed position.

Another and/or alternative non-limiting object of the present disclosure is the provision of a method for stabilizing a limb of an individual, the method further comprising the step of, prior to positioning the portion of the limb on the first inner surface of the first member, depositing a filler material on the first inner surface of the first member and the second inner surface of the second member, wherein the filler material is configured to conform to a shape of the limb.

Another and/or alternative non-limiting object of the present disclosure is the provision of a method for stabilizing a limb of an individual, the method wherein the filler material includes silicon, urethane, latex rubber, epoxy resin, polyethylene foam, or combinations thereof.

Another and/or alternative non-limiting object of the present disclosure is the provision of a method for stabilizing a limb of an individual, the method further comprising the step of, subsequent to pivoting the orthosis device into the closed position, securing the first member to the second member through the coupler arrangement that includes first and second couplers that are spaced apart from one another.

Another and/or alternative non-limiting object of the present disclosure is the provision of a method for stabilizing a limb of an individual, the method wherein the coupler arrangement includes one or more of clasps, snaps, buttons, hooks, adhesives, zipper, magnets, hook and loop fasteners, mushroom connectors, or combinations thereof.

Another and/or alternative non-limiting object of the present disclosure is the provision of a method for stabilizing a limb of an individual, the method wherein the coupler arrangement includes a hinge, and wherein the hinge extends at least partially or fully along a longitudinal length of the orthosis device.

Another and/or alternative non-limiting object of the present disclosure is the provision of a method for stabilizing a limb of an individual, the method wherein the hinge is a living hinge, a piano hinge, a butt hinge, or a leaf hinge.

Another and/or alternative non-limiting object of the present disclosure is the provision of a method for stabilizing a limb of an individual, the method wherein the hinge includes an adhesive at least partially deposited on a contact surface of the hinge, wherein the adhesive is a repositionable adhesive, a cohesive adhesive, or combinations thereof.

Another and/or alternative non-limiting object of the present disclosure is the provision of a method for stabilizing a limb of an individual, the method further comprising the step of, forming one or more markers at predetermined locations on the first member, the second member, or the first and second members to guide an individual in removing a portion of the orthosis device.

Another and/or alternative non-limiting object of the present disclosure is the provision of a method for stabilizing a limb of an individual, the method further comprising the step of, subsequent to pivoting the orthosis device into the closed position, forming an outer shell is substantially cylindrical, elliptical, cuboidal, spherical, or triangular.

These and other objects and advantages will become apparent to those skilled in the art upon reading and following the description taken together with the accompanying drawings.

### BRIEF DESCRIPTION OF THE DRAWINGS

The accompanying drawings, which are incorporated into and form a part of the specification, schematically illustrate one or more example implementations of the disclosed technology and, together with the general description given above and detailed description given below, serve to explain the principles of the disclosed subject matter, and wherein:

FIG. 1 is an illustration of the front view of a splint in accordance with a non-limiting embodiment of the present invention.

FIG. 2 is an illustration of the rear view of a splint in accordance with a non-limiting embodiment of the present invention.

FIG. 3 is an illustration of the rear view of a splint in accordance with another non-limiting embodiment of the present invention.

FIG. 4 is an illustration of the rear view of a splint in an open position in accordance with a non-limiting embodiment of the present invention.

FIG. 5 is an illustration of the top view of a finger splint in accordance with a non-limiting embodiment of the present invention.

FIG. 6 is an illustration of the bottom view of a splint in accordance with a non-limiting embodiment of the present invention.

FIG. 7 is an illustration of the top view of a splint in an open position displaying the method of dispersing inner material into said finger splint in accordance with a non-limiting embodiment of the present invention.

FIG. 8 is another illustration of the top view of a splint in an open position filled with an inner material in accordance with a non-limiting embodiment of the present invention.

FIG. 9 is another illustration of the top view of a splint in an open position filled with an inner material molding a form of human finger in accordance with a non-limiting embodiment of the present invention.

FIG. 10 is an illustration of the perspective view of a finger splint circumferentially surrounding a human finger in accordance with a non-limiting embodiment of the present invention.

### DETAILED DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

One or more implementations of the subject application will now be described with reference to the attached figures, wherein like reference numerals are used to refer to like elements throughout.

While the present invention is directed toward a splint for use with any appendage, limb, finger, or toe, the following description will be described as a “finger splint.” One having skill in the art would recognize that the application of the invention could be utilized on a leg, an arm, a portion of an arm, a portion of a leg, a hand, a portion of a hand, a foot, a portion of a foot, one or more toes, or one or more fingers, or any other part of a body of a human or animal needing such treatment.

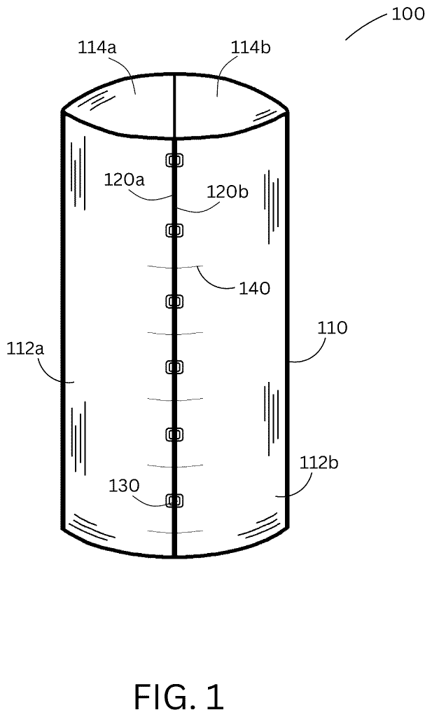

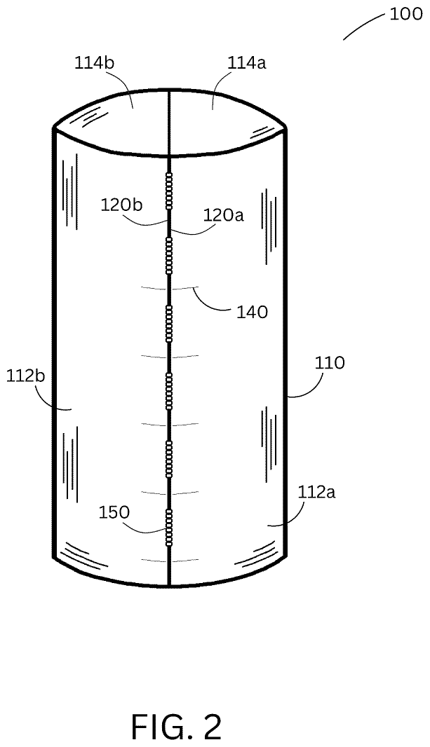

Referring to FIGS. 1-2, a first embodiment of the invention is illustrated. Disclosed is a finger splint 100 comprising of an outer shell 110 shaped cylindrically, divided in perfect half lengthwise, and may be open on at least one of its two half bases 114a and 114b. The outer shell 110 is generally hollow and open at its proximal base and includes two half portions 112a and 112b (e.g., first and second members), optionally substantially equal, and may be of any size or proportionality suitable for the ingress and egress of a human finger or thumb in and out of the outer shell 110. The outer shell 110 may be shaped as a right circular cylinder, oblique cylinder, or elliptical cylinder and is connected at the hinge 150 acting as a clamshell designed to open and close. In some non-limiting embodiments, the outer shell 110 may or may not be transparent and may be made from High Density Polyethylene (HDPE), Low Density Polyethylene (LDPE), Polyethylene Terephthalate (PET), Polypropylene (PP), and/or any other solid material designed to retain its shape. The outer shell 110 may be any shape and any thickness. In the present non-limiting embodiment, the outer shell 110 is a cylindrical shape formed by two meeting halves. In other embodiments, the outer shell 110 may be cuboidal, spherical, triangular, polygonal, a series of rings, or any other shape. In some embodiments, the outer shell 110 is formed from three or more separate portions that fit together to form a protective body.

Still referring to FIGS. 1-2, the hinge 150 (e.g., first coupler) is attached at the rear abutting edge portions 120a and 120b along a portion or all of the longitudinal length of the half portions 112a and 112b and is designed to provide stability and connect the two half portions 112a and 112b to form the outer shell 110 and allow the two half portions 112a and 112b to rotate or pivot or otherwise move at a fixed point or points. The hinge 150 is configured to maintain edge portions 120a and 120b together or in a generally constant spacing from one another when the outer shell is moved between the open and closed position and when the outer shell is in the open and closed position. The hinge 150 may comprise a living hinge, piano hinge, butt hinge, leaf hinge, or any other hinge designed to connect and pivot two half portions 112a and 112b together. In one non-limiting embodiment, the hinge is molded to one or both of half portions 112a and 112b. In another non-limiting embodiment, the hinge is a living hinge that is molded to one or both of half portions 112a and 112b. The hinge 150 may have any number of contact points between the half portions 112 and 114 and, in some non-limiting embodiments, may be made from High Density Polyethylene (HDPE), Low Density Polyethylene (LDPE), Polyethylene Terephthalate (PET), Polypropylene (PP), and/or any other solid material that has flexibility and can withstand repeated opening and closing. In other embodiments, there is no hinge 150 and rather the outer shell 110 is comprised of two separate half portions that are completely separate from one another. In such an alternative arrangement, the two half portions 112a and 112b are connected together by first and second couplers that are both absent a hinge.

Still referring to FIGS. 1-2, attached to the outer shell 110 at the front abutting edge portions 120a and 120b are any number of clasps 130 (e.g., second coupler) made of any size or shape and placed in any position along the vertical front abutting edge portions 120a and 120b and along the longitudinal length of the two half portions 112a and 112b. In some non-limiting embodiments, the clasp or clasps 130 may be made from exact materials of the outer shell 110 or made any other material that can securely attach to the outer shell 110 surface and provide ample latch pressure between the half portions 112a and 112b. The clasps 130 may be comprised of a button form, hook-and-eye, snap lock, zipper, magnets, hook and loop fastener, mushroom connector, or male and female snap connector or any other form of connection mechanism and may or may not be molded to form on each of the half portions 112a and 112b. In other embodiments, the clasps 130 may be replaced by or supplemented with a strap or tape that surrounds the body of the outer shell 110. As one having skill in the art could recognize, the clasps 130 can be replaced by any known mechanism to ensure a continuous closed connection between the halves of the outer shell 110, such as a strap, buttons, magnets, zipper, hook and loop fastener, mushroom connectors, hooks, tape, of any other known method or component.

In some non-limiting embodiments, optional trim markers 140 are horizontally molded on the half portions 112a and/or 112a or otherwise positioned in horizontally molded on the half portions 112a and/or 112a, providing a guide for customizability to the finger splint 100. It is to be appreciated that any number of trim markers 140 may be located any distance apart and positioned anywhere and in any direction on the outer shell 110. The trim markers 140 act as guides to allow a user to trim (e.g. cut using scissors, knife, etc.) and decrease the vertical dimension of the finger splint 100 to form fit to any number of unique finger or thumb sizes. The trim makers 140 may be simple markings on the outer shell 110 and/or may be indentations set into the body of the outer shell 110. In some embodiments the trim markers 140 may be partial apertures in the body of the outer shell 110. The markers can optionally be colored markings.

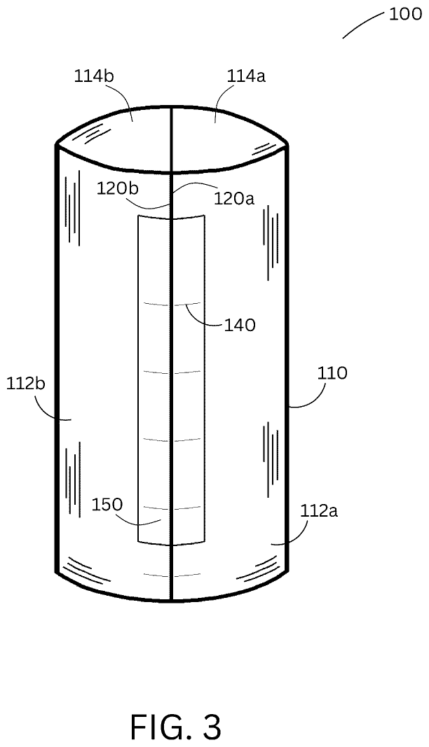

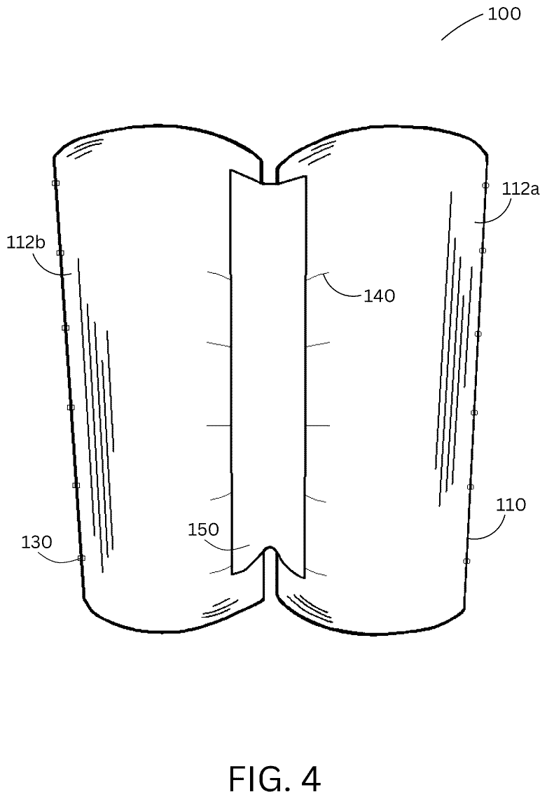

FIGS. 3-4 illustrate another embodiment of the invention in which the hinge 150 may be comprised of a self-adhesive strip and may or may not have a pre-manufactured acrylic piano hinge. The self-adhesive strip hinge 150 may or may not be pre-applied during manufacture and may require the user to apply said self-adhesive strip as the necessary length may not be known and may require customization to be trimmed to suitable length. The self-adhesive strip hinge 150 may be made of a repositionable/remoistenable polyvinylchloride (PVC) backing and/or a cohesive non-corrosive rubber-based adhesive or any other materials that provide ample flexibility and secure adhesion to the half portions 112a and 112b of the outer shell 110 while still able to withstand repeated opening and closing. The self-adhesive strip hinge 150 may or may not be premolded into the half portions 112a and 112b.

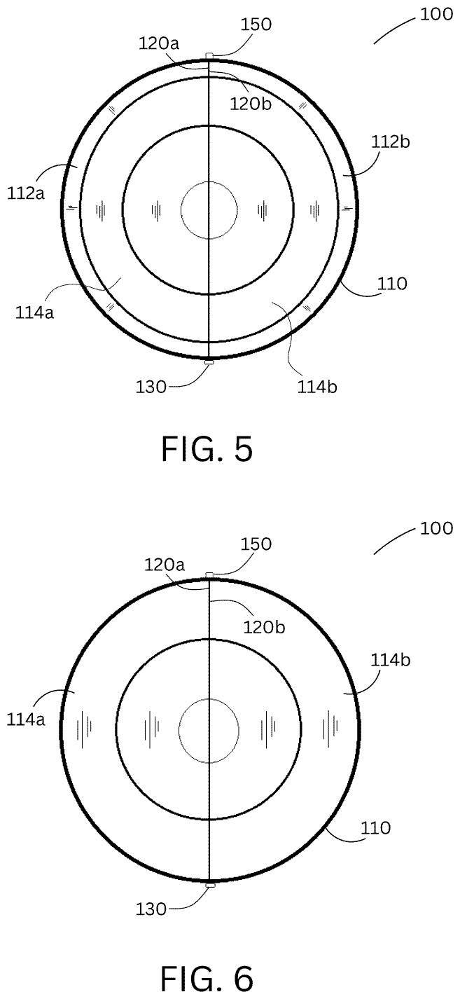

Referring to FIGS. 5-6 are illustrations of the top and bottom view of the finger splint 100, respectively. The outer shell 110 in its overall configuration in a closed state may be cylindrically formed, or formed of any other shape of any dimension, and of a material having a thickness providing substantial rigidity and is completely open at the proximal base. The full distal base is comprised of half bases 114a and 114b of the outer shell 110 and the cylinder base is formed when the half portions 112a and 112b and the half bases 114a and 114b are in a fully closed position having abutting edge portions 120a and 120b. In alternative non-limiting embodiments, the distal base, when the outer shell 110 is engaged in a closed position having abutting edge portions 120a and 120b, may be entirely open and not exist to allow additional air, or may be partially (e.g., 10-99.99% closed and all values and ranges therebetween) or be entirely closed, or either of the half bases 114a and 114b of the half portions 112a and 112b may be open or closed creating a partially open and partially closed distal base. In other embodiments the proximal base may have lateral portions disposed inward toward the center of the outer shell 110. The lateral portions may meet together at certain portions and have a center aperture for receiving the finger of a user. The lateral portions may be stiff and rigid made from the same material as the outer shell 110 or may be soft flanges of a rubber or foam material.

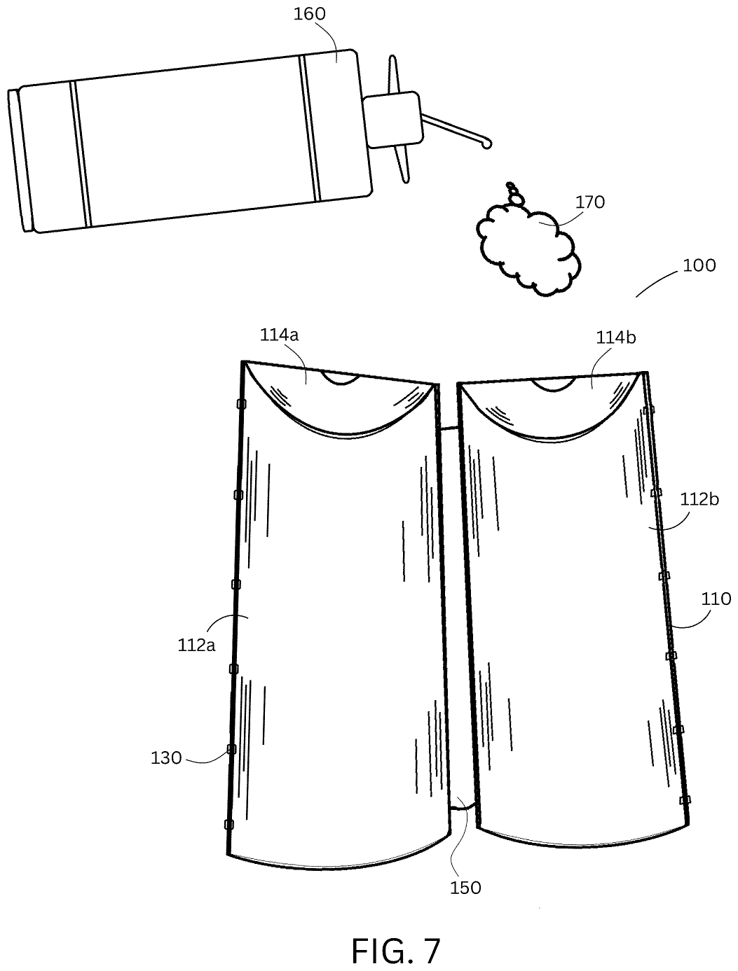

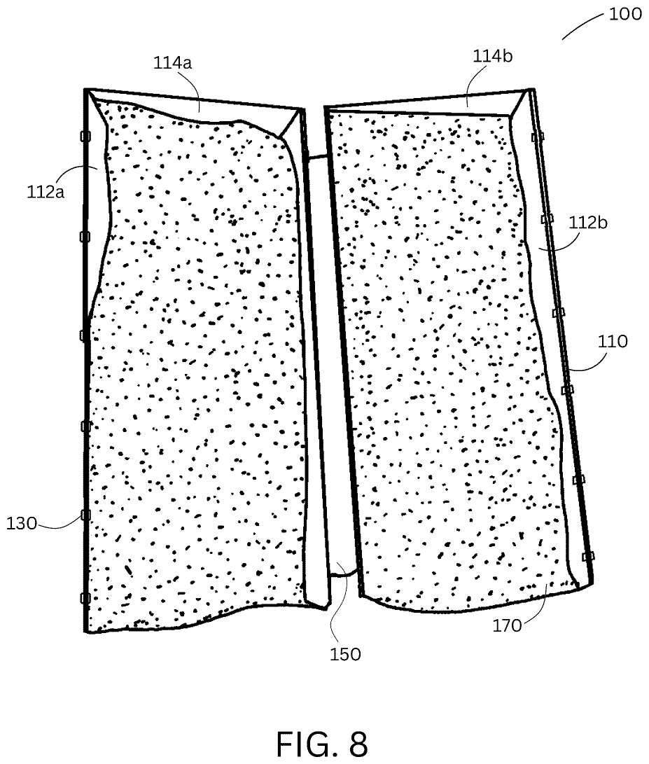

FIGS. 7-8 are illustrations of the top view of the finger splint 100 in a fully open position demonstrating the method of introducing the inner material 170 into the hollow inner cavities of the half portions 112a and 112b of the outer shell 110. In this non-limiting embodiment, the inner material 170 is dispersed from the spray bottle 160 or other type of dispenser in a generally even manner and generally to not exceed the vertical apex of the half portions 112a and 112b of the outer shell 110. As can be appreciated, the inner material 170 can be applied to the hollow inner cavities of the half portions 112a and 112b of the outer shell 110 by other means (e.g., pouring the inner material 170 into one or both of the hollow inner cavities of the half portions 112a and 112b, adhesively connecting or otherwise securing one or more pieces of inner material 170 into one or both of the hollow inner cavities of the half portions 112a and 112b). The inner material 170 may be made from a skin safe silicon, urethane, or latex rubber, epoxy resin, polyethylene foam and/or any other moldable material (e.g., moldable thermoplastic material, etc.) that hardens to a semi-firm state in a short time duration. The inner material 170 may be poured, sprayed, injected, or set of any material and dispensed from any container, and may become harder from any liquid material or softer from any harder material and may be hard or soft.

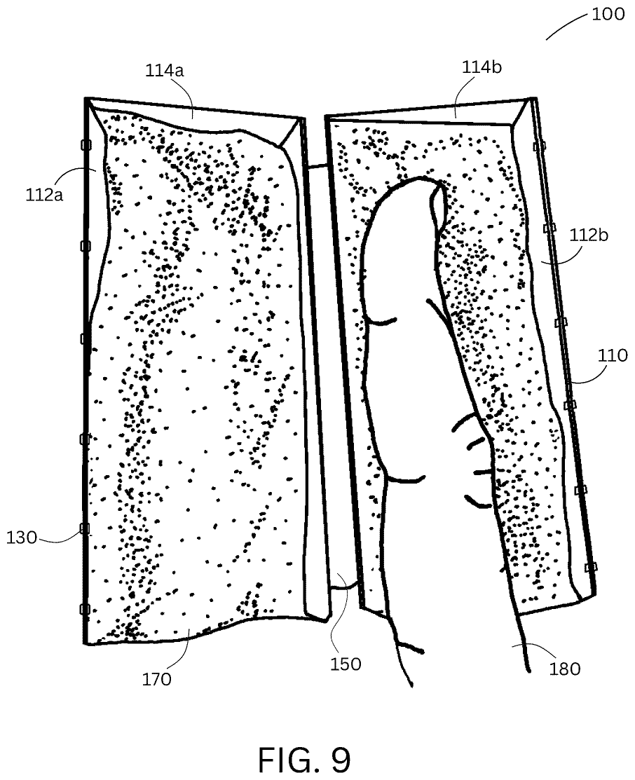

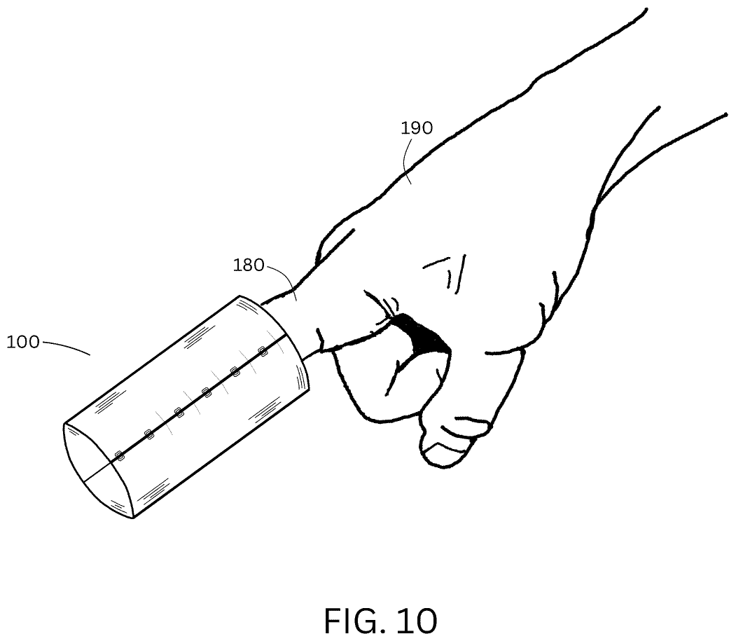

Referring to FIGS. 9-10 the non-limiting method of use is further illustrated in the present embodiment. Once the inner material 170 has been inserted into the half portions 112a and 112b, a user may place an injured or ailing finger, thumb, or toe into the half portion 112a or 112b. The user may optionally use a thin, conforming, protective covering over the human digit 180 to keep it unsoiled from the inner material 170. The inner material 170 is made from any material suitable for taking an impression and once the human digit 180 is placed firmly in the finger splint 100, it is closed circumferentially around the human digit 180. With the outer shell 110 engaged in a closed position having abutting edge portions 120a and 120b, the human digit 180 is molded of oneself and is splinted circumferentially on both sides, palmarly, dorsally, and the at the tip of said digit. Once the inner material has set, cured, dried, etc. over any period of time, the finger splint 100 can be opened, the protective digit material removed from the human digit, and the splint is ready to provide treatment by conforming to the finger, thumb, and/or toe in order to hold member securely. Referring to FIG. 10, one non-limiting utilization of the finger splint 100 is presented. If the thin, conforming, protective covering over the human digit is a thin, conforming, protective covering can be disposed of after the human digit is removed from the finger splint.

The finger splint 100 may have any other combination of elements or components added on without diverting from the scope of the invention. In one embodiment, the distal base comprising the half bases 114a and 114b may be detached before or after the process of setting the inner material 170 and may be able to be reattached. In another embodiment, the hinge 150 may be located at the distal base and open and close vertically along the y-axis. The finger splint 100 may or may not contain a kit of necessary components to enable a user to complete the method of preparing the orthosis device. The kit may or may not contain one or any number of components including an outer shell 110, hinge and clasping mechanism, protective digit material, inner material 170 contained in any form of container, trimming apparatus, and instructions describing the method of use.

When in use, the inner material 170 typically mostly (e.g., fills 50-99.00 vol. % and all values and ranges therebetween) or fully fills the entire cavity created by the outer shell 110. While one skilled in the art would recognize that voids could form in the inner material 170 after it is placed in the inner cavity of the outer shell 110 without altering the scope of the invention, in one non-limiting embodiment there are few or no gaps or spaces between the digit 180 and the inner material 170, or the inner material 170 and the outer shell 110 (e.g., 0-10 percent of the volume of the inner cavity [and all values and ranges therebetween] includes gaps or voids when the digit is in the inner cavity and the orthosis device is in the closed position). At the very least, there is typically one space in the inner cavity where there are no gaps or spaces between the digit 180 and the inner material 170, or the inner material 170 and the outer shell 110.

It will be appreciated that any of a variety of additional or alternative steps may be included in a method of using, assembling/preparing, and/or disassembling the finger splint 100.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. These and other modifications of the preferred embodiments as well as other embodiments of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

The description of embodiments and examples has been presented for purposes of illustration and description. It is not intended to be exhaustive or limiting to the forms described. Numerous modifications are possible in light of the teachings herein. Some of those modifications have been discussed, and others will be understood by those skilled in the art. The embodiments were chosen and described in order to illustrate principles of various embodiments as are suited to particular uses contemplated. The scope is, of course, not limited to the examples set forth herein, but can be employed in any number of applications and equivalent devices by those of ordinary skill in the art.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. An orthosis device comprising:
(a) a first member having a first inner surface that is configured to receive a first portion of a limb;
(b) a second member having a second inner surface that is configured to receive a second portion of the limb; and
(c) a coupler arrangement that is positioned along a portion or all of a longitudinal length of said first member and second members to enable said first and second members to be transitionable between an open position and a closed position.

2. The orthosis device as defined in claim 1, further comprising a filler material that is configured to conform to a shape of the limb, and wherein said filler material is disposed on said first inner surface of said first member and/or said second inner surface of said second member.

3. The orthosis device as defined in claim 2, wherein the filler material includes silicon, urethane, latex rubber, epoxy resin, polyethylene foam, moldable thermoplastic, or combinations thereof.

4. The orthosis device as defined in claim 1, wherein said coupler arrangement includes a hinge.

5. The orthosis device as defined in claim 4, wherein said hinge is a living hinge, a piano hinge, a butt hinge, or a leaf hinge.

6. The orthosis device as defined in claim 4, wherein the hinge includes an adhesive that is at least partially deposited on a contact surface of the hinge, and wherein said adhesive is a repositionable adhesive, a cohesive adhesive, or combinations thereof.

7. The orthosis device as defined in claim 1, further comprising one or more markers formed at predetermined locations on the first member, the second member, or the first and second members, wherein said one or more markers guide an individual in removing a portion of said orthosis device.

8. The orthosis device as defined in claim 1, wherein said coupler arrangement includes first and second couplers that are configured to secure said first member to said second member when said orthosis device is in the closed position, and wherein said first and second couplers are spaced apart from one another.

9. The orthosis device as defined in claim 8, wherein said second coupler includes one or more of clasps, snaps, magnets, zipper, hook and loop fastener, mushroom connectors, buttons, hooks, adhesives, or combinations thereof.

10. The orthosis device as defined in claim 1, wherein said first member and said second member are configured to form an outer shell when said orthosis device is in said closed position, and wherein said outer shell is substantially cylindrical, elliptical, cuboidal, spherical, or triangular.

11. A method for stabilizing a limb of an individual, said method comprising the steps of:
(a) providing an orthosis device comprising:
(i) a first member having a first inner surface;
(ii) a second member having a second inner surface; and
(iii) a coupler arrangement that couples said first member to said second member, and wherein said coupler arrangement is positioned along a portion or the full longitudinal length of said first and said members;
(b) positioning said orthosis device in an open position;
(c) positioning a portion of the limb on said first inner surface of said first member and/or said second inner surface of said second member; and
(d) moving said orthosis device into a closed position and using said coupler arrangement to at least partially secure said first and second members in said closed position.

12. The method as defined in claim 11, further comprising the step of, prior to positioning said portion of said limb on said first inner surface of said first member and/or said second inner surface of said second member, depositing a filler material on said first inner surface of said first member and/or said second inner surface of said second member, and wherein said filler material is configured to at least partially conform to a shape of said limb.

13. The method as defined in claim 12, wherein the filler material includes silicon, urethane, latex rubber, epoxy resin, polyethylene foam, moldable thermoplastic, or combinations thereof.

14. The method as defined in claim 11, further comprising the step of, subsequent to moving said orthosis device to said closed position, securing the said first member to said second member through said coupler arrangement, and wherein said coupler arrangement includes first and second couplers, and wherein said first and second couplers are spaced apart from one another.

15. The method as defined in claim 14, wherein said second coupler includes one or more of clasps, snaps, buttons, hooks, zippers, hook and loop fasteners, mushroom connects, magnets, adhesives, or combinations thereof.

16. The method as defined in claim 11, wherein said first coupler includes a hinge.

17. The method as defined in claim 16, wherein said hinge is a living hinge, a piano hinge, a butt hinge, or a leaf hinge.

18. The method as defined in claim 16, wherein said hinge includes an adhesive that is at least partially deposited on a contact surface of said hinge, wherein said adhesive is a repositionable adhesive, a cohesive adhesive, or combinations thereof.

19. The method as defined in claim 14, further comprising the step of, forming one or more markers at predetermined locations on said first member and/or said second member to guide an individual in removing a portion of said orthosis device.

20. The method as defined in claim 14, further comprising the step of, subsequent to moving said orthosis device into said closed position, forming an outer shell is substantially cylindrical, elliptical, cuboidal, spherical, or triangular.
